(19) United States
(12) Patent Application Publication
(10) Pub. No.: US 2009/0215769 A1
(43) Pub. Date: Aug. 27, 2009

(54) Use of soluble guanylate cyclase activators for the treatment of Raynaud's Phenomenon

(75) Inventors: Thomas Krahn, Hagen (DE); Johannes-Peter Stasch, Solingen (DE); Gerrit Weimann, Köln (DE); Wolfgang Thielemann, Wuppertal (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/988,991

(22) PCT Filed: Jul. 4, 2006
(86) PCT No.: PCT/EP2006/006501

§ 371 Date: Feb. 27, 2009

(30) Foreign Application Priority Data
Jul. 16, 2005 (DE) 10 2005 033 370.2
Oct. 6, 2005 (DE) 10 2005 047 945.6

(51) Int. Cl. A61K 31/5377; A61K 31/195; A61K 31/505

(52) U.S. Cl. 514/234.2; 514/567; 514/256; 514/231.5; 514/236.8

(57) ABSTRACT

The present invention relates to a method for prevention or treatment of primary and secondary Raynaud's phenomenon comprising administration of an effective amount of a compound selected from compounds of formulae I-IV, and to pharmaceutical compositions containing these compounds.

The present invention relates to the use of compounds of the formulae I-VI for manufacturing a pharmaceutical for the treatment of primary and secondary Raynaud's phenomenon.

Raynaud's phenomenon describes episodic phases of digital vasoconstriction with narrowing of digital arteries, precapillary arteriols and cutaneous arteriovenous shunts. The clinical signs are episodic painful ischemic phases in the fingers, followed by a cyanotic blue discoloration and finally a phase of reactive hyperemia. Possible inducers are cold stimuli and stress reactions. A distinction is moreover made between primary, uncomplicated Raynaud's phenomenon with an excessive physiological response of the extremity arteries to cold stimuli, and a secondary Raynaud's phenomenon with a basic systemic disorder which is a rheumatoid disease or one of the connective tissue diseases such as scleroderma, Sjögren's syndrome, systemic lupus erythematosus, rheumatoid arthritis, polymyositis or dermatomyositis. Secondary Raynaud's phenomenon is moreover frequently associated with tissue necroses on the fingers, ulcerations and gangrene development.

The pathophysiological basis is assumed to be an altered vascular tone of the digital arteries and arterioles. It has additionally been observed firstly that the expression of NO and prostaglandins is reduced and secondly that the production of endothelin is increased.

The therapeutic approaches to date have been confined supportively to the avoidance of inducing stimuli such as cold and stress, nicotine abuse and vasoconstrictive medicaments (e.g. ergotamine, clonidine, sympathomimetics), and symptomatically the administration of various vasodilators (e.g. calcium channel blockers, a, receptor blockers, nitrate products and prostaglandin derivatives). No studies have yet been carried out on large groups of patients. Often only collections of cases and anecdotal reports on the various substances exist, sometimes with contradictory results. The individual vasodilating agents show response rates of only about 50%. Side effects are frequent: thus, calcium channel blockers and a, receptor blockers show systemic hypotensions. Intravenous prostaglandins have not to date been authorized for the indication of Raynaud's disease, and studies with various prostaglandin derivatives (iloprost, beraprost, cicaprost) have not been able to show any clinical benefits for this pathological condition.

One of the most important cellular transmission systems in the mammalian cells is cyclic guanosine monophosphate (cGMP). Together with nitric oxide (NO), which is released from the endothelium and transmits hormonal and mechanical signals, it forms the NO/cGMP system. Guanylate cyclases catalyze the biosynthesis of cGMP from guanosine triphosphate (GTP). The representatives of this family which are known to date can be divided into two groups both according to structural features and according to the nature of the ligands: the particulate guanylate cyclases which can be stimulated by natriuretic peptides, and the soluble guanylate cyclases which can be stimulated by NO. The soluble guanylate cyclases consist of two subunits and very probably contain one heme per heterodimer, which is part of the regulatory center. This has a central importance for the mechanism of activation. NO is able to bind to the iron atom of the heme and thus distinctly increase the activity of the enzyme. Heme-free preparations by contrast cannot be stimulated by NO. CO is also able to attach to the central iron atom of heme, but the stimulation by CO is distinctly less than that by NO.

Through the production of cGMP and the regulation, resulting therefrom, of phosphodiesterases, ion channels and protein kinases, guanylate cyclase plays a crucial part in various physiological processes, in particular in the relaxation and proliferation of smooth muscle cells, in platelet aggregation and adhesion and in neuronal signal transmission, and in disorders caused by an impairment of the aforementioned processes. Under pathophysiological conditions, the NO-cGMP system may be suppressed, possibly leading for example to high blood pressure, platelet activation, increased cellular proliferation, endothelial dysfunction, atherosclerosis, angina pectoris, heart failure, thromboses, stroke, sexual dysfunction and myocardial infarction.

A possible treatment of such diseases which is independent of NO and aims at influencing the cGMP signaling pathway in organisms is a promising approach because of the high efficiency and few side effects to be expected.

Compounds such as organic nitrates, whose effect is based on NO, have to date been exclusively used for the therapeutic stimulation of soluble quanylate cyclase. NO is produced by bioconversion and activates soluble guanylate cyclase by acting on the central iron atom of heme. Besides the side effects, the development of tolerance is one of the crucial disadvantages of this mode of treatment.

It has now been found that stimulators of soluble guanylate cyclase are particularly suitable for the therapy of microangiopathies. Compared with the prior art, the compounds of the invention of formulae I to VI listed below have improved pharmacodynamic properties: they have vasodilating effects irrespective of the NO produced endogeneously in the arterial capillary system even when there is severe endothelial damage. In addition, stimulators of soluble guanylate cyclase enhance the effect of the endogenously produced NO.

Compound (I) corresponds to the following formula:

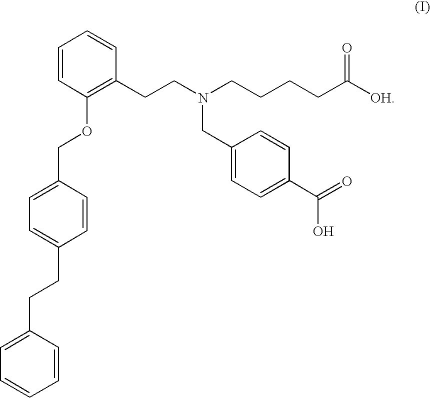

Compound (I), the preparation and use thereof as pharmaceutical have been disclosed in WO 01/19780.

Compound (II) corresponds to the following formula:

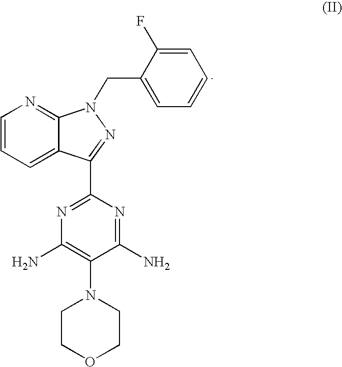

Compound (II), the preparation and use thereof as pharmaceutical have been disclosed in WO 00/06569.

Compound (III) corresponds to the following formula:

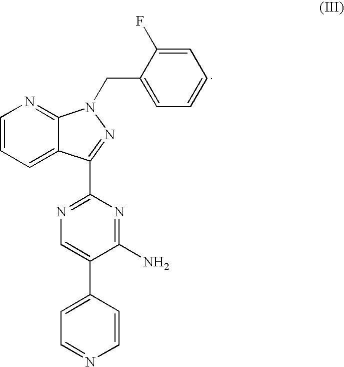

Compound (III), the preparation and use thereof as pharmaceutical have been disclosed in WO 00/06569 and WO 02/42301.

Compound (IV) corresponds to the following formula:

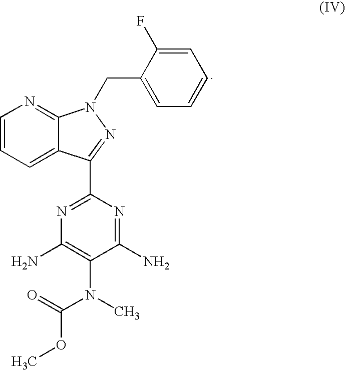

Compound (IV), the preparation and use thereof as pharmaceutical have been disclosed in WO 00/06569 and WO 03/095451.

Compound (IVa) corresponds to the following formula:

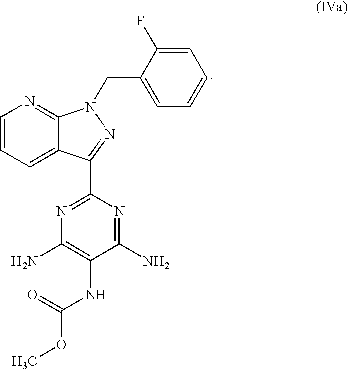

Compound (IVa), the preparation and use thereof as pharmaceutical have been disclosed in WO 00/06569 and WO 03/095451.

Compound (V) corresponds to the following formula:

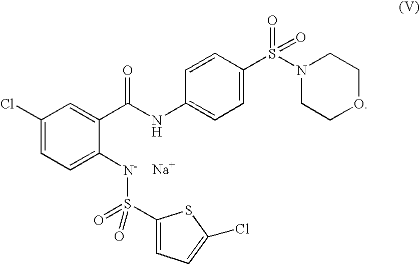

Compound (VI) corresponds to the following formula:

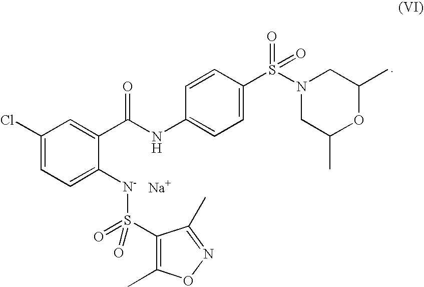

Compounds (V) and (VI), the preparation and use thereof as pharmaceutical have been disclosed in WO 00/0285 1.

The present invention relates to the use of compounds of the formulae (I-VI) and the salts, hydrates, hydrates of the salts thereof for the manufacture of a medicament for the treatment of primary and secondary Raynaud's phenomenon.

An additional exemplary embodiment of the present invention includes the procedure for the prophylaxis and/or treatment of pulmonary hypertension by using at least one of the compounds of the formulae (I-VI).

The present invention further relates to pharmaceuticals comprising at least one compound of the invention and at least one or more further active ingredients, especially for the treatment and/or prophylaxis of the aforementioned disorders.

The compounds of the invention may have systemic and/or local effects. They can for this purpose be administered in a suitable way, such as, for example, by the oral, parenteral, pulmonary, nasal, sublingual, lingual, buccal, rectal, dermal, transdermal, conjunctival or otic route or as implant or stent.

The compounds of the invention can be administered in suitable administration forms for these administration routes.

Administration forms suitable for oral administration are those which function according to the state of the art and deliver the compounds of the invention in a rapid and/or modified way, and which contain the compounds of the invention in crystalline and/or amorphized and/or dissolved form, such as, for example, tablets (uncoated or coated tablets, for example with coatings which are resistant to gastric juice or dissolve slowly or are insoluble and which control the release of the compound of the invention), tablets which rapidly disintegrate in the mouth, or films/wafers, films/lyophilizates, capsules (for example hard or soft gelatin capsules), sugar-coated tablets, granules, pellets, powders, emulsions, suspensions, aerosols or solutions.

Parenteral administration can take place with avoidance of an absorption step (e.g. intravenous, intraarterial, intracardiac, intraspinal or intralumbar) or with inclusion of an absorption (e.g. intramuscular, subcutaneous, intracutaneous, percutaneous or intraperitoneal). Administration forms suitable for parenteral administration are, inter alia, injection and infusion preparations in the form of solutions, suspensions, emulsions, lyophilizates or sterile powders.

Examples suitable for other administration routes are medicinal forms for inhalation (inter alia powder inhalers, nebulizers), nasal drops, solutions, sprays; tablets for lingual, sublingual or buccal administration, films/wafers or capsules, suppositories, preparations for the ears or eyes, vaginal capsules, aqueous suspensions (lotions, shaking mixtures), lipophilic suspensions, ointments, creams, transdermal therapeutic systems (such as, for example, patches), milk, pastes, foams, dusting powders, implants or stents.

The compounds of the invention can be converted into the stated administration forms. This can take place in a manner known per se by mixing with inert, non-toxic, pharmaceutically suitable excipients. These excipients include, inter alia, carriers (for example microcrystalline cellulose, lactose, mannitol), solvents (e.g. liquid polyethylene glycols), emulsifiers and dispersants or wetting agents (for example sodium dodecyl sulfate, polyoxysorbitan oleate), binders (for example polyvinylpyrrolidone), synthetic and natural polymers (for example albumin), stabilizers (e.g. anti-oxidants such as, for example, ascorbic acid), colors (e.g. inorganic pigments such as, for example, iron oxides) and masking tastes and/or odors.

The present invention further relates to pharmaceuticals which comprise at least one compound of the invention, normally together with one or more inert, non-toxic, pharmaceutically suitable excipients, and to the use thereof for the aforementioned purposes.

It has generally proved advantageous to administer amounts of about 0.01 to 5000 mg/kg, preferably about 0.5 to 1000 mg/kg, of body weight per day to achieve effective results.

It may nevertheless be necessary to deviate from the stated amounts, in particular as a function of body weight, administration route, individual behavior towards the active ingredient, type of preparation and time or interval over which administration takes place. Thus, it may in some cases be sufficient to make do with less than the aforementioned minimum amount, whereas in other cases the stated upper limit must be exceeded. Where larger amounts are administered, it may be advisable to divide them into a plurality of single doses over the day.

The formulations can moreover comprise, appropriate for the intervention, active substance between 0.1 and 99% active ingredient, in a suitable manner 25-95% in the case of tablets and capsules and 1-50% in the case of liquid formulations, i.e. the active ingredient should be present in amounts sufficient to achieve the stated dose range.

1. A method for prevention or treatment of primary and secondary Raynaud's phenomenon comprising administering an effective amount of a compound selected from compounds of the formulae (I-VI) 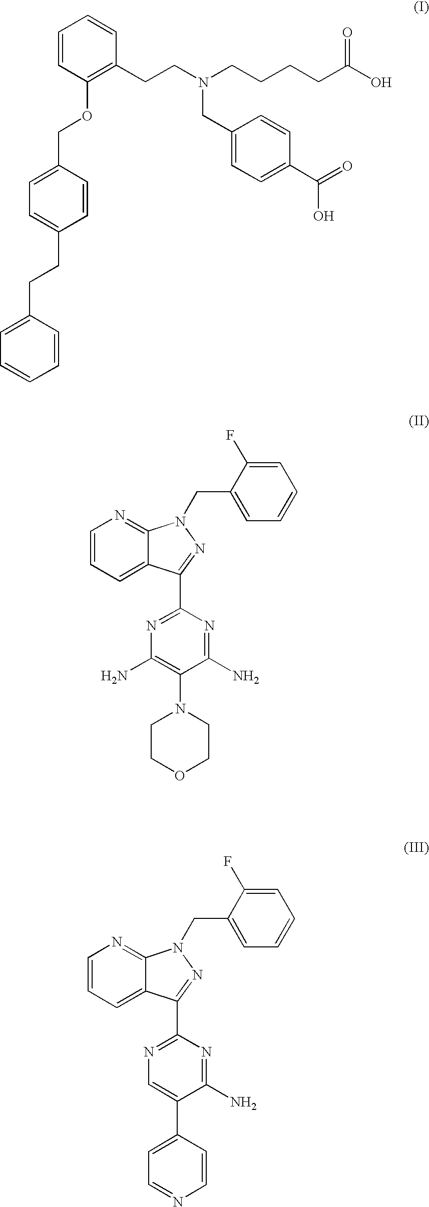 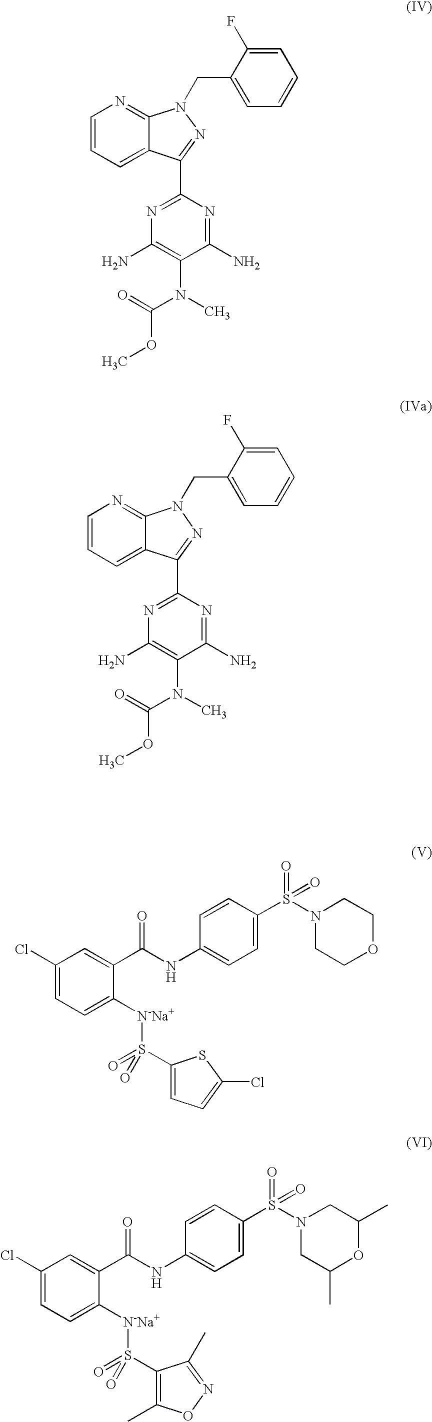
or a salt, hydrate, or hydrate of a salt thereof.

2. The method as claimed in claim 1, in which the compound is administered as an oral dosage form.

3. The method as claimed in claim 1, in which the compound is given intravenously.

4. The method as claimed in claim 1, in which the compound is preventive.

5. A pharmaceutical composition for treating primary and secondary Raynaud's phenomenon, which comprises at least one substance as described in claim 1.
